(19) United States
(12) Patent Application Publication
(10) Pub. No.: US 2024/0225993 A1
(43) Pub. Date: Jul. 11, 2024

(54) HAIR COMPOSITE FOR HAIR THICKENING AND HAIR GROWTH

(71) Applicant: Tanya Payton, Newport News, VA (US)

(72) Inventor: Tanya Payton, Newport News, VA (US)

(21) Appl. No.: 18/093,714

(22) Filed: Jan. 5, 2023

(51) Int. Cl. A61K 8/92; A61K 8/67; A61Q 7/00

(52) U.S. Cl. CPC A61K 8/922; A61K 8/678; A61Q 7/00; A61K 2800/59

(57) ABSTRACT

A hair treatment grease configured to be applied to a scalp, is disclosed. The hair treatment grease includes at least one oil base comprising at least one part of one or more of tea tree oil, jojoba oil, myrrh oil, frankincense oil, and/or peppermint oil. The hair treatment grease includes at least one fat-soluble antioxidant formed into a mixture with the at least one oil base.

### FIELD OF THE INVENTION

This disclosure relates to a composite for use on the scalp and hair of a person. Specifically, this disclosure relates to a composite that facilitates the thickening and growth of hair.

### BACKGROUND

Hair loss is a concern that is suffered by men, women, children, and people of all ages. Hair loss is often suffered from exposure to stress, health complications, and the use of unstable chemicals. Additionally, it is medically known that individuals can lose up to 100 hairs from their scalp every day. However, as humans grow older, hair loss can increase substantially as people get older.

With hair loss, normally follows baldness at different stages. Baldness typically refers to excessive hair loss from your scalp. Hereditary hair loss with age is the most common cause of baldness. Some people prefer to let their hair loss run its course untreated and unhidden. Others may cover it up with hairstyles, makeup, hats or scarves. And still others choose one of the treatments available to prevent further hair loss or restore growth.

Accordingly, embodiments of the invention, provide significant advantages while overcoming the above-described and other disadvantages, as will now be described.

### SUMMARY

A hair treatment grease configured to be applied to a scalp, is disclosed. The hair treatment grease includes at least one oil base comprising at least one part of one or more of tea tree oil, jojoba oil, myrrh oil, frankincense oil, and/or peppermint oil. The hair treatment grease includes at least one fat-soluble antioxidant formed into a mixture with the at least one oil base.

In one embodiment, the great is configured to provide moisturization to the scalp.

In one embodiment, the moisturization of the scalp reduces dandruff on the scalp.

In one embodiment, the mixing of the mixture creates a friction and bonding reaction that results in heating.

In one embodiment, the mixtures include at least one-part Jamaican black castor oil, and coconut oil.

In one embodiment, the mixture further includes almond oil.

This summary is provided merely for purposes of summarizing some example embodiments, so as to provide a basic understanding of some aspects of the subject matter described herein.

Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, FIGURES, and Claims.

### BRIEF DESCRIPTION OF THE DRAWINGS

Disclosed herein are embodiments of low energy motorized platform of solar panels. This description includes drawings, wherein:

FIG. 1 depicts a method of preparing the hair treatment grease, in accordance with an example.

Elements in the figures are illustrated for simplicity and clarity and have not been drawn to scale. For example, the dimensions and/or relative positioning of some of the elements in the FIGURES may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein.

### DETAILED DESCRIPTION

Some detailed example embodiments are disclosed herein. However, specific structural and functional details disclosed herein are merely representative for purposes of describing example embodiments. Example embodiments may, however, be embodied in many alternate forms and should not be construed as limited to only the embodiments set forth herein.

The invention may be embodied in other specific forms without departing from the spirit of essential characteristics thereof. The present embodiments therefore to be considered in all respects as illustrative and not restrictive, the scope of the invention being indicated by the appended claims rather than by the foregoing description, and all changes which come within the meaning and range of equivalency of the claims are therefore intended to be embraced therein.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The following description is not to be taken in a limiting sense but is made merely for the purpose of describing the general principles of exemplary embodiments. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The preferred embodiment herein described is not intended to be exhaustive or to limit the invention to the precise form disclosed. It is chosen and described to explain the principles of the invention and its application and practical use to enable others skilled in the art to follow its teachings.

The term “hair composite” encompasses many types of combination of chemicals, products and applications for the hair and scalp. For example, applications of greases, chemical relaxer compositions, shampoos, conditioners, hair-rinses, hair lotions, hair gels, mouse-type products, and sprays can all be forms of implementation. A hair treatment composite is characterized by its ability to provide a cosmetic benefit to the hair, in relation to thickness, length, and healthiness.

A hair treatment grease configured to be applied to a scalp, is disclosed. The hair treatment grease includes at least one oil base comprising at least one part of one or more of tea tree oil, jojoba oil, myrrh oil, frankincense oil, and/or peppermint oil. The hair treatment grease includes at least one fat-soluble antioxidant formed into a mixture with the at least one oil base.

Accordingly, the mixture is configured to be heated in at least two stages selected from stages comprising mixing, physical handling, application to the scalp, and bonding with the scalp. The heating of the mixture stimulates the scalp and activates hair follicle regrowth.

The benefit of the use of jojoba oil is created from jojoba seeds and contains about 50% of liquid wax which is mainly used to create a moisturizing coating for cosmetic preparations by creating a protective film over the skin that keeps in moisture. Jojoba oil provides a broad spectrum of fatty acids, as well as triglycerides which have good compatibility with the natural sebum in the human skin. Jojoba oil further has significant analgesic, antipyretic, anti-inflammatory, antioxidant, anti-bacterial and anti-parasitic properties.

Similarly, tea tree oil has been used as a pure essential oil and as an active ingredient in products to provide antimicrobial, antiseptic, and anti-inflammatory properties. Many of the same bacteria that tea tree oil combats on the face can be found on the scalp which is known to cause dandruff and irritation. Tea tree oil is utilized to transform a person's hair, starting at the scalp. Its antimicrobial, antibacterial, and anti-inflammatory properties provide for a potent treatment for dandruff, that improves overall scalp health, which is directly related to increasing overall healthy hair growth. The use of tea tree oil as several beneficial properties that include antioxidant and antimicrobial properties which relieve itchy conditions and pain in and on the scalp. Using tea tree oil also provides the benefit or reduced inflammation which is in on the leading causes of hair loss. Tea tree oil also regulates oil production resulting in a healthier scalp and overall healthiness of hair.

Frankincense oil is used in order to help slow and prevent hair loss, while also stimulating hair growth when applied topically to the scalp. Frankincense can often times contain Vitamin E, which help to nourish and strengthen the hair from within.

Frankincense oil also acts to inhibit the body's production of a chemical compound known as alpha 5-reductase, which occurs when testosterone gets blocked in the pores under your scalp. The excess amount of testosterone reacts with the hair follicles in a person's scalp, which results in the reductase forming as the result of this reaction. The reductase, when reaching unhealth levels, clogs up the pores of the hair follicles and starves them of essential nutrients, which prevents the release of natural oils that keep the hairs moisturized, causing the hairs to become weaker and break or fall out.

In some instances, frankincense can be used in order to conduct heat in a hot oil treatment. The heating conductivity of frankincense in combination with another essential oil such as coconut oil, olive oil, and almond oil, is activated upon being mixed, stirred or blended. The oil can then be handled in order to maintain or increase the heat conductivity, and then massaged into a person's scalp in circular motions. The heat conductivity causes the scalp to be treated with moisture, as well as allow necessary inhibiting qualities that kill hair stunting bacteria, returning the scalp to a hair growth state, which allows for healthy hair that includes thickness and length.

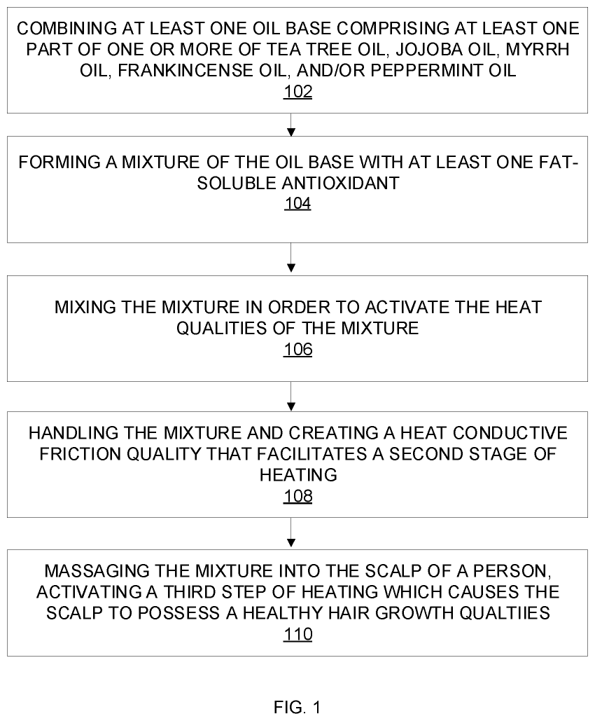

FIG. 1 depicts a method of implementing a preparation of the hair treatment grease. The method of preparation includes a first step of combining at least one oil base comprising at least one part of one or more of tea tree oil, jojoba oil, myrrh oil, frankincense oil, and/or peppermint oil. The preparation further includes a second step forming a mixture of the oil base with at least one fat-soluble antioxidant. The third step includes mixing the mixture in order to activate the heat qualities of the mixture. The fourth step includes handling the mixture and creating a heat conductive friction quality that facilitates a second stage of heating. The fifth step includes massaging the mixture into the scalp of a person, activating a third step of heating which causes the scalp to possess healthy hair growth qualities.

Example embodiments having thus been described, it will be obvious that the same may be varied in many ways. Such variations are not to be regarded as a departure from the intended spirit and scope of example embodiments, and all such modifications as would be obvious to one skilled in the art are intended to be included within the scope of the following claims.

Although the present invention has been described in terms of various embodiments, it is not intended that the invention be limited to these embodiments. Modification within the spirit of the invention will be apparent to those skilled in the art.

It is additionally noted and anticipated that although the device is shown in its most simple form, various components and aspects of the device may be differently shaped or modified when forming the invention herein. As such those skilled in the art will appreciate the descriptions and depictions set forth in this disclosure or merely meant to portray examples of preferred modes within the overall scope and intent of the invention and are not to be considered limiting in any manner. While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the scope of the invention.

What is claimed is:

1. A hair treatment grease configured to be applied to a scalp comprising:
at least one oil base comprising at least one part of one or more of tea tree oil, jojoba oil, myrrh oil, frankincense oil, and/or peppermint oil; and
at least one fat-soluble antioxidant formed into a mixture with the at least one oil base;
wherein the mixture is configured to be heated in at least two stages selected from stages comprising mixing, physical handling, application to the scalp, and bonding with the scalp; and
wherein the heating of the mixture stimulates the scalp and activates hair follicle regrowth.

2. The hair treatment grease of claim 1, wherein the great is configured to provide moisturization to the scalp.

3. The hair treatment grease of claim 1, wherein the moisturization of the scalp reduces dandruff on the scalp.

4. The hair treatment grease of claim 1, wherein the mixing of the mixture creates a friction and bonding reaction that results in heating.

5. The hair treatment grease of claim 1, further comprising at least one-part Jamaican black castor oil, and coconut oil.

6. The hair treatment grease of claim 1, further comprising almond oil.

7. The hair treatment grease of claim 1, wherein the composition is in a spray form.

8. The hair treatment grease of claim 1, wherein the mixture forms a gelatin, or cream base.

9. The hair treatment great of claim 1, wherein the at least one fat-soluble antioxidant comprises Vitamin E.

10. A method of implementing a preparation of the hair treatment grease, the method comprising:
combining at least one oil base comprising at least one part of one or more of tea tree oil, jojoba oil, myrrh oil, frankincense oil, and/or peppermint oil;
forming a mixture of the oil base with at least one fat-soluble antioxidant;
mixing the mixture in order to activate one or more heat qualities of the mixture;
handling the mixture and creating a heat conductive friction quality that facilitates a second stage of heating; and
massaging the mixture into the scalp of a person, activating a third stage of heating which causes the scalp to possess healthy hair growth qualities.
